(19) United States
(12) Patent Application Publication
(10) Pub. No.: US 2019/0060135 A1
(43) Pub. Date: Feb. 28, 2019

(54) Method and Apparatus for Manufacturing an Absorbent Article

(71) Applicant: The Procter & Gamble Company, Cincinnati, OH (US)

(72) Inventor: Paul Anthony Kawka, Cleves, OH (US)

(21) Appl. No.: 16/048,701

(22) Filed: Jul. 30, 2018

Related U.S. Application Data
(60) Provisional application No. 62/551,932, filed on Aug. 30, 2017.

(51) Int. Cl. A61F 13/15; B29C 65/74; B29C 65/00

(52) U.S. Cl. CPC A61F 13/15772; A61F 13/15764; A61F 13/15739; A61F 13/4963; B29C 66/95; A61F 2013/15796; B29C 65/74

(57) ABSTRACT

A method and apparatus for assembling an absorbent article. A converting apparatus may be used to assemble an absorbent article such that features on each component of the absorbent article are positioned with respect to features present on joining components of the absorbent article. One or more sensors are used to detect registration features based on the features of each component of the absorbent article. The sensor communicates with a controller to change the speed and position of the components to assemble the absorbent article such that features are positioned with respect to one another.

### FIELD

The present disclosure relates to methods and apparatuses for manufacturing absorbent articles, and more particularly, methods and apparatuses for advancing, cutting, and bonding portions of an absorbent article.

### BACKGROUND

Along an assembly line, various types of articles, such as, diapers, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from advancing web or webs are combined with other individual components created from other advancing web or webs. Webs of material and component parts used to manufacture diapers may include: backsheets, topsheets, leg cuffs, waist bands, absorbent core components, front and/or back ears, fastening components, and various types of elastic webs and components such as leg elastics, barrier leg cuff elastics, stretch side panels, and waist elastics. Once the desired component parts are assembled, the advancing webs and component parts are subjected to a final knife cut to separate the webs into discrete diapers or other absorbent articles.

Absorbent articles are increasing including one or more features such as graphics, bonds, and other visually discernable features. Each component of the absorbent article such as the backsheet, topsheet, waistband, front ears, back ears, fastening components, and landing zone may include one or more features. Often these features are designed such that features of one component compliment or correspond to features of another component. Further, as the absorbent article is being manufactured these components need to be place with respect to one another such that the features are present in a desired location in the final product. Traditionally, one or more registration marks are placed on the components such that a sensor can identify the registration mark and cut, transfer, and/or bond the component part as desired. However, these registration marks require an additional mark to be placed on the component in addition to the desired features. Further, these marks are required to be in areas that are uninhibited by other features so that the registration marks can be identified by the sensor. Alternatively, relatively large portions of components are left “blank” or, stated another way, an area is intentionally left absent of any features so that a sensor can identify this blank area. Thus, manufactures must leave areas intentionally devoid of features and these blank areas may lead to blank areas in the final absorbent article or increased difficulty during the manufacturing process to conceal these blank areas. It is undesirable to obtain a final product having features on various components that fail correspond to one another or a final product have relatively large blank areas. Therefore, it would be beneficial to provide a process and apparatus that does not require additional registration marks or blank areas and that produces a final product having the desired features and those features that are strategically placed to correspond to other features.

### SUMMARY

Aspects of the present disclosure may include a method for assembling a multi-piece absorbent article. The method may include: feeding a chassis to a first assembly station at a first velocity, the chassis comprising a topsheet, a backsheet joined to the topsheet, and an absorbent core positioned between the topsheet and the backsheet; feeding an ear substrate to a second assembly station at a second velocity, wherein the second velocity is different than the first velocity; feeding a securement member substrate to the second assembly station at a third velocity, wherein the third velocity is different than the second velocity, wherein the securement member substrate comprises first registration features and the ear web comprises second registration features; identifying the first registration features using a first sensor; cutting the securement member substrate into individual securement members based on a position of the first registration features; identifying the second registration features using a second sensor; bonding the individual securement members to the ear substrate at the second assembly station, wherein each of the individual securement members is positioned with respect to an individual feature on the ear substrate; cutting the ear substrate into individual ear pieces; and bonding the individual ear pieces to the chassis at the first assembly station to form a multi-piece absorbent article.

In some embodiments, a method for assembling a multi-piece absorbent article may include: feeding a chassis to a first assembly station at a first velocity, the chassis comprising a topsheet, a backsheet joined to the topsheet, and an absorbent core positioned between the topsheet and the backsheet; feeding an ear substrate to a second assembly station at a second velocity; feeding a securement member substrate to the second assembly station at a third velocity, wherein the securement member substrate comprises first registration features and the ear substrate comprises second registration features; detecting positions of the first registration features using a first sensor; cutting the securement member substrate into individual securement members based on the position of each of the first registration features; detecting positions of the second registration features using a second sensor; bonding the individual securement members to the ear substrate at the second assembly station based on the position of each of the second registration features; feeding the ear substrate with the individual securement members bonded thereto to the first assembly station, wherein the ear substrate with the individual securement members comprises third registration features; detecting positions of the third registration features using a third sensor; cutting the ear substrate into individual ear pieces based on the positions of each of the third registration features, wherein the chassis comprises fourth registration features; detecting the positions of the fourth registration features using a fourth sensor; and bonding the individual ear pieces to the chassis based on a position of the fourth registration features at the first assembly station to form a multi-piece absorbent article.

In some embodiments, a method for assembling a multi-piece absorbent article may include: feeding a chassis to a first assembly station at a first velocity, the chassis comprising a topsheet, a backsheet joined to the topsheet, and an absorbent core positioned between the topsheet and the backsheet; feeding an ear substrate to a second assembly station at a second velocity; feeding a securement member substrate to the second assembly station at a third velocity, wherein the securement member substrate comprises first registration features and second registration features; teaching a first sensor to recognize the first registration features; identifying the first registration features using the first sensor; cutting the securement member substrate into individual securement members based on a position of the first registration features; teaching the first sensor to recognize the second registration features; identifying the second registration features using the first sensor; cutting the securement member substrate into individual securement members based on a position of the second registration features; switching between the first sensor recognizing the first registration features and the first sensor recognizing the second registration features; bonding the individual securement members to the ear substrate at the second assembly station, wherein each of the individual securement members is positioned with respect to an individual feature on the ear substrate; cutting the ear substrate into individual ear pieces; and bonding the individual ear pieces to the chassis at the first assembly station to form a multi-piece absorbent article.

### BRIEF DESCRIPTION OF THE DRAWINGS

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of an example absorbent article of the present disclosure that is a sanitary napkin;

FIG. 7 is a schematic, side view of a converting apparatus;

FIG. 8 is a schematic, side view of a converting apparatus;

FIG. 9 is a plan view of a portion of a securement member substrate;

FIG. 10 is a plan view of a portion of an ear substrate;

FIG. 11 is a plan view of a portion of a chassis;

FIG. 12A is a plan view of an ear;

FIG. 12B is a plan view of an ear;

FIG. 12C is a plan view of a portion of a chassis including an ear;

FIG. 13 is a schematic, side view of a converting apparatus;

FIG. 14 is a plan view of a portion of a securement member substrate; and

FIG. 15 is a plan view of a portion of an ear substrate.

### DETAILED DESCRIPTION

The following definitions may be useful in understanding the present disclosure:

“Absorbent article” is used herein to refer to consumer products whose primary function is to absorb and retain soils and wastes. “Diaper” is used herein to refer to an absorbent article generally worn by infants and incontinent persons about the lower torso. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

“Joined” is used herein to encompass configurations whereby an element is directly secured to another element by affixing the element directly to the other element, and configurations whereby an element is indirectly secured to another element by affixing the element to intermediate member(s) which in turn are affixed to the other element.

As used herein, the term “graphic” refers to images or designs that are constituted by a figure (e.g., a line(s)), a symbol or character, a color difference or transition of at least two colors, or the like. A graphic may include an aesthetic image or design that can provide certain benefit(s) when viewed. A graphic may be in the form of a photographic image. A graphic may also be in the form of a 1-dimensional (1-D) or 2-dimensional (2-D) bar code or a quick response (QR) bar code. A graphic design is determined by, for example, the color(s) used in the graphic (individual pure ink or spot colors as well as built process colors), the sizes of the entire graphic (or components of the graphic), the positions of the graphic (or components of the graphic), the movements of the graphic (or components of the graphic), the geometrical shapes of the graphic (or components of the graphics), the number of colors in the graphic, the variations of the color combinations in the graphic, the number of graphics printed, the disappearance of color(s) in the graphic, and the contents of text messages in the graphic.

“Longitudinal” means a direction running substantially perpendicular from a waist edge to a longitudinally opposing waist edge of an absorbent article when the article is in a flat out, uncontracted state, or from a waist edge to the bottom of the crotch, i.e. the fold line, in a bi-folded article. Directions within 45 degrees of the longitudinal direction are considered to be “longitudinal.” “Lateral” refers to a direction running from a longitudinally extending side edge to a laterally opposing longitudinally extending side edge of an article and generally at a right angle to the longitudinal direction. Directions within 45 degrees of the lateral direction are considered to be “lateral.”

“Substrate” is used herein to describe a material which is primarily two-dimensional (i.e. in an XY plane) and whose thickness (in a Z direction) is relatively small (i.e. 1/10 or less) in comparison to the substrate's length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

“Nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

“Machine direction” (MD) is used herein to refer to the direction of material flow through a process. In addition, relative placement and movement of material can be described as flowing in the machine direction through a process from upstream in the process to downstream in the process.

“Cross direction” (CD) is used herein to refer to a direction that is not parallel with, and usually perpendicular to, the machine direction.

“Pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed).

“Pre-fastened” refers herein to pant diapers manufactured and provided to consumers in a configuration wherein the front waist region and the back waist region are fastened or connected to each other as packaged, prior to being applied to the wearer. As such pant diapers may have a continuous perimeter waist opening and continuous perimeter leg openings designed for infant or adult wearers. As discussed in more detail below, a diaper pant can be preformed by various techniques including, but not limited to, joining together portions of the diaper using refastenable and/or permanent closure members (e.g., seams, heat bonds, pressure welds, adhesives, cohesive bonds, mechanical fasteners, etc.). In addition, pant diapers can be preformed anywhere along the circumference of the waist region (e.g., side fastened or connected, front waist fastened or connected, rear waist fastened or connected).

The present disclosure relates to methods and apparatuses for assembling absorbent articles, and in particular, to methods and apparatuses for controlling the relative placement of advancing substrates and discrete components in diaper converting lines. The diapers may each include a chassis. The chassis may include a topsheet, a backsheet, and an absorbent core disposed between the topsheet and the backsheet. The chassis may also have a first end region and an opposing second end region separated from each other by a central region. During the assembly process, opposing end regions of the chassis are connected with the elastic belts in the form of first and second continuous elastic laminates. Alternatively, during the assembly process, at least one of the first end region and the second end region are connected with ear pieces. In controlling the relative placement of these elements during the assembly process, a controller may change the machine direction speed and/or position of certain elements and cross direction speed and/or position of other elements such as the advancing substrates and components in order to help achieve proper placement. The controller may affect such changes in speeds and positions based on the detection of registration features. In some configurations, the first and/or second elastic laminates, securement members, ears, and chassis may include registration features.

During the assembly process, the registration features are detected, and a controller may change the machine direction speeds of the advancing substrate and/or may change the cross directional and/or machine direction position of the advancing substrate. It is to be appreciated that the speed changes discussed herein may be transient changes or steady step changes. With a transient change, an object or substrate advancing at a first speed may be temporarily accelerated or decelerated to a second speed, and then decelerated or accelerated back to the first speed. With steady step change, an object or substrate advancing at a first speed may be accelerated or decelerated to a second speed.

For example, as discussed below, a first continuous substrate, such as a substrate of interconnected chassis, may advance in a machine direction at a first speed. The first continuous substrate includes features arranged along the machine direction. In addition, a second continuous substrate, such as a continuous length of ears, may advance in the machine direction at a second speed. The second continuous substrate includes second features arranged along the machine direction. In addition, a third continuous substrate, such as a continuous length of securement members, may advance in the machine direction at a third speed. The third continuous substrate includes features arranged along the machine direction. The features of each of the first, second, and third continuous substrates may be detected by a sensor. The sensor may be taught to identify registration features based on the features of the substrates. The advancing third continuous substrate may be cut into discrete securement members based on the identification of the registration features by the sensor. Based on detections of the registration features, a controller may then adjust the speed of the second continuous substrate and/or the cross direction position of the second continuous substrate and the controller may adjust the speed of the third continuous substrate and/or shift the third continuous substrate in the cross direction. In addition, a controller may alter the speed of the cutting device. The securement members may be placed on the second continuous substrate based on the registration features.

Similarly, the advancing second continuous substrate may be cut into discrete ears based on the identification of the registration features by the sensor. Based on detections of the registration features, a controller may then adjust the speed of the third continuous substrate and/or the cross direction position of the third continuous substrate and the controller may adjust the speed of the first continuous substrate and/or shift the first continuous substrate in the cross direction. In addition, a controller may alter the speed of the cutting device or other process equipment. The ears may be positioned on the first continuous substrate in a desired location based on the registration features. Subsequently, the first continuous substrate may be cut in a direction substantially parallel to the cross direction to form discrete absorbent articles.

As discussed in more detail below, features or portions of features on various substrates may be used by one or more sensors to identify registration features. These registration features may be used to make adjustments the assembly process. For example, the registration features may be used to combine the securement members with the advancing ear substrate in such a way to maximize the aesthetic appearances of the features on assembled products. Some features may be configured to appear as a design that appears to extend contiguously across combined diaper components, such as the front elastic belt, back elastic belt, ears, securement members, landing zones, and/or chassis. Thus, in some converting configurations, continuous substrates used to form some components of the absorbent article may include portions of such features. And some other components, such as a backsheet or topsheet, may include other portions of such features. Therefore, during the assembly process, the components may be assembled such that the graphic portions are combined to provide the appearance of contiguous designs that extend across more than one component.

As previously mentioned, the processes and apparatuses discussed herein may be used in the manufacture of different types of absorbent articles. To help provide additional context to the subsequent discussion of the process embodiments, the following provides a general description of absorbent articles that may be manufactured in accordance with the methods and apparatuses disclosed herein.

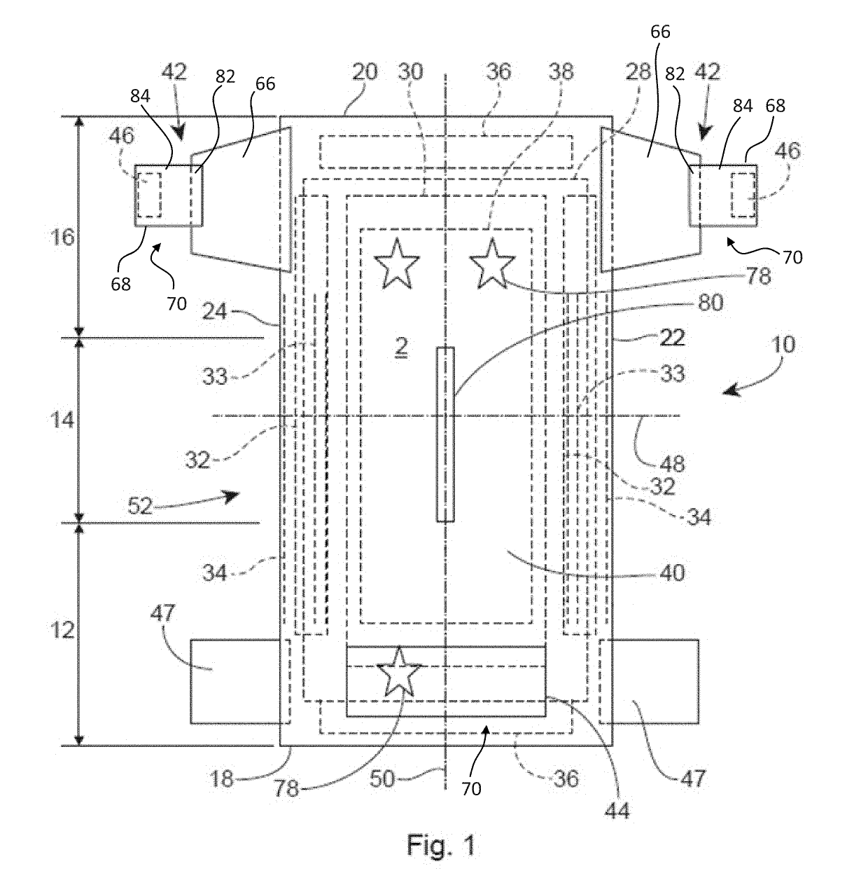

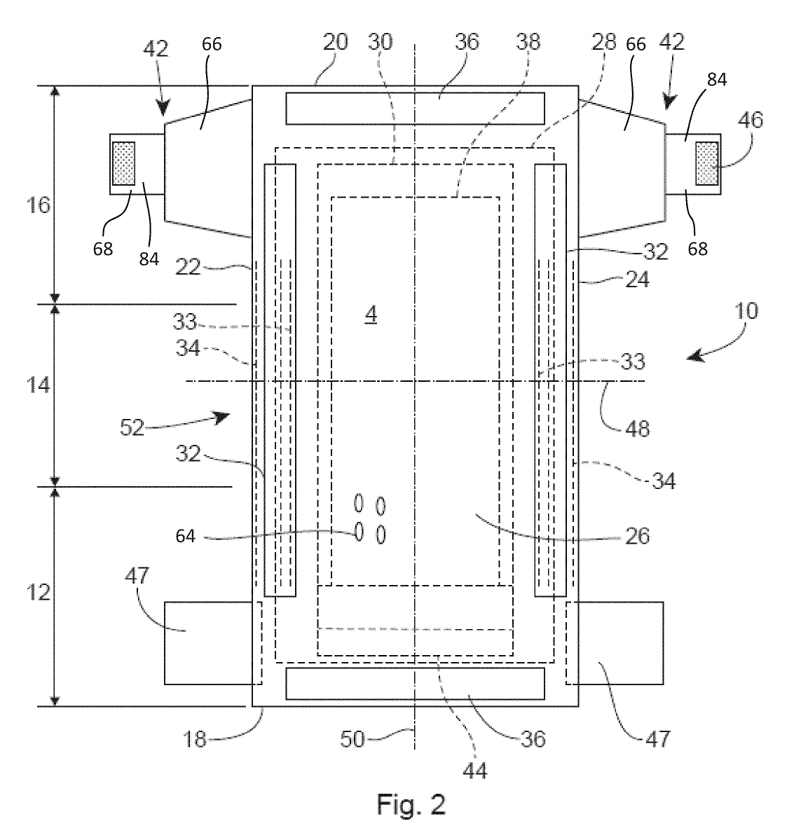

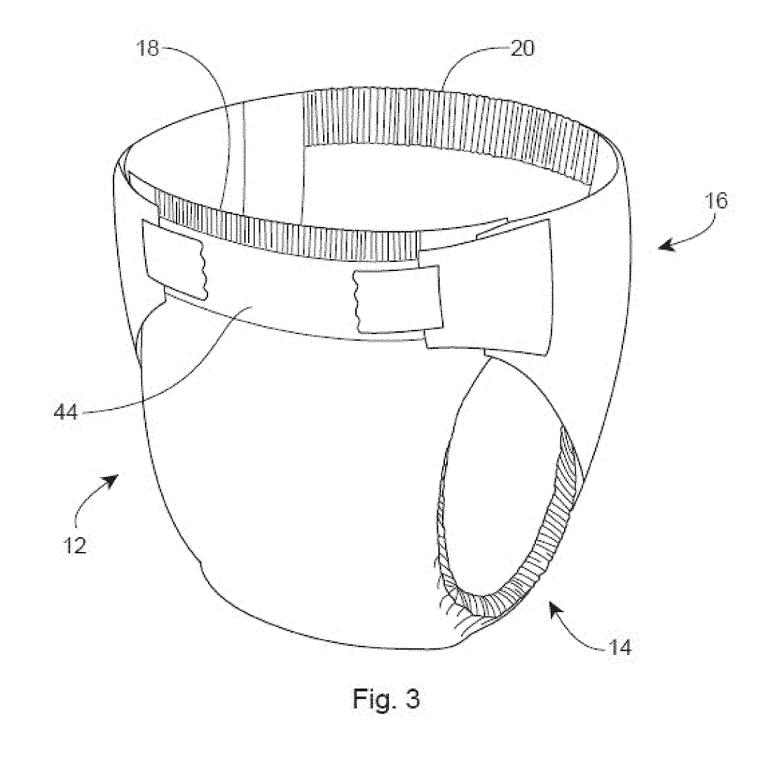

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

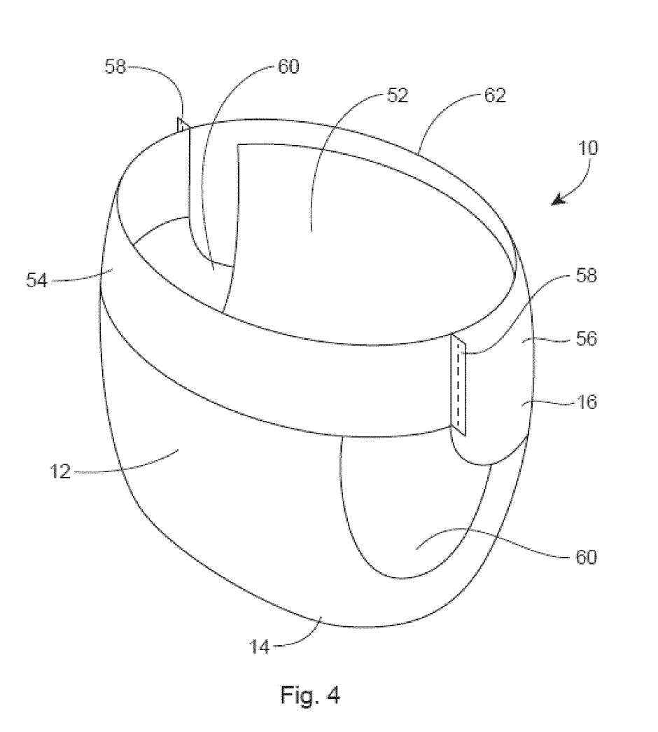

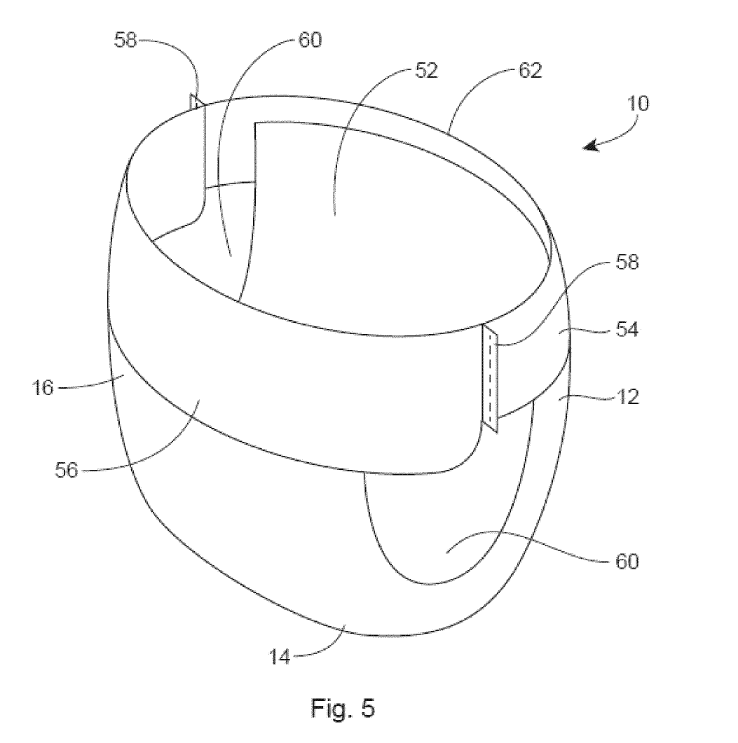

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137. Referring to FIGS. 4 and 5, an example absorbent article 10 in the form of a pant is illustrated. FIG. 4 is a front perspective view of the absorbent article 10. FIG. 5 is a rear perspective view of the absorbent article 10. Elements of FIGS. 4 and 5 having the same reference number as described above with respect to FIGS. 1-3 may be the same element (e.g., absorbent core 30). The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges and of the front belt 54 may be joined to side edges of the back belt 56, respectively, to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

The front and back belts 54 and 56 may comprise front and back inner belt layers and front and back outer belt layers having an elastomeric material (e.g., strands or a film (which may be apertured)) disposed at least partially therebetween. The elastic elements or the film may be relaxed (including being cut) to reduce elastic strain over the absorbent core 30 or, may alternatively, run continuously across the absorbent core 30. The elastics elements may have uniform or variable spacing therebetween in any portion of the belts. The elastic elements may also be pre-strained the same amount or different amounts. The front and/or back belts 54 and 56 may have one or more elastic element free zones where the chassis 52 overlaps the belts 54, 56. In other instances, at least some of the elastic elements 68 may extend continuously across the chassis 52.

The front and back inner belt layers and the front and back outer belt layers may be joined using adhesives, heat bonds, pressure bonds or thermoplastic bonds. Various suitable belt layer configurations can be found in U.S. Pat. Appl. Pub. No. 2013/0211363 A1.

Front and back belt end edges may extend longitudinally beyond the front and back chassis end edges or they may be co-terminus. The front and back belt side edges may extend laterally beyond the chassis side edges 22 and 24. The front and back belts 54 and 56 may be continuous (i.e., having at least one layer that is continuous) from belt side edge to belt side edge. Alternatively, the front and back belts 54 and 56 may be discontinuous from belt side edge to belt side edge, such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer and the back outer belt layer may be separated from each other, such that the layers are discrete or, alternatively, these layers may be continuous, such that a layer runs continuously from the front belt end edge to the back belt end edge. This may also be true for the front and back inner belt layers—that is, they may also be longitudinally discrete or continuous. Further, the front and back outer belt layers may be longitudinally continuous while the front and back inner belt layers are longitudinally discrete, such that a gap is formed between them.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt seams; or, alternatively, adjacent to the seams in the manner described in U.S. Pat. No. 9,498, 389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 64), may have any suitable three-dimensional features, and/or may have a plurality of embossments (e.g., a bond pattern). The topsheet may be apertured by overbonding a material and then rupturing the overbonds through ring rolling, such as disclosed in U.S. Pat. No. 5,628,097, to Benson, et al., issued on May 13, 1997 and disclosed in U.S. Pat. Appl. Publication No. 2016/0136014 A1 to Arora, et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet. The topsheet may comprise graphics such that depth perception is created as described in U.S. Pat. No. 7,163,528.

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features.

As used herein, the term “absorbent core” 30 refers to the component of the absorbent article 10 having the most absorbent capacity and that comprises an absorbent material. In some instances, absorbent material 72 may be positioned within a core bag or a core wrap 74. The absorbent material may be profiled or not profiled, depending on the specific absorbent article. The absorbent core 30 may comprise, consist essentially of, or consist of, a core wrap, absorbent material 72, and glue enclosed within the core wrap. The absorbent material may comprise superabsorbent polymers, a mixture of superabsorbent polymers and air felt, only air felt, and/or a high internal phase emulsion foam. In some instances, the absorbent material may comprise at least 80%, at least 85%, at least 90%, at least 95%, at least 99%, or up to 100% superabsorbent polymers, by weight of the absorbent material. In such instances, the absorbent material may free of air felt, or at least mostly free of air felt. The absorbent core periphery, which may be the periphery of the core wrap, may define any suitable shape, such as rectangular “T,” “Y,” “hour-glass,” or “dog-bone” shaped, for example, such as disclosed in U.S. Pat. No. 8,939,957 and U.S. Patent Publication No. 2012/0277702 A1. An absorbent core periphery having a generally “dog bone” or “hour-glass” shape may taper along its width towards the crotch region 14 of the absorbent article 10.

The absorbent core 30 may have areas having little or no absorbent material 72, where a wearer-facing surface of the core bag 74 may be joined to a garment-facing surface of the core bag 74. These areas having little or no absorbent material may be referred to as channels. These channels can embody any suitable shapes and any suitable number of channels may be provided. In other instances, the absorbent core may be embossed to create the impression of channels. Many other absorbent cores with or without channels are also within the scope of the present disclosure. Exemplary cores are described in U.S. Pat. Nos. 5,599,335; 5,562,646; 5,669,894; and 6,790,798 as well as U.S. Patent Publication Nos. 2004/0158212 A1 and 2004/0097895 A1.

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14. Example leg cuffs may include those described in U.S. Pat. Nos. 3,860,003; 4,909,803; 4,695,278; 4,795,454; 4,704,115; 4,909,803; U.S. Patent Publication No. 2009/0312730 A1; and U.S. Patent Publication No. 2013/0255865 A1.

Referring to FIGS. 1 and 2, the absorbent article 10 may comprise one or more elastic waistbands 36. The elastic waistbands 36 may be positioned on the garment-facing surface 2 or the wearer-facing surface 4. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article. The elasticized waistband may be constructed in a number of different configurations including those described in U.S. Pat. Nos. 4,515,595 and 5,151,092. Further, the waistband may be constructed as disclosed in U.S. Publication Nos. 2012/0330262 A1; 2012/0330263 A1; and 2012/0330264 A1 such that the waistband works in combination with the leg cuffs to provide improved fit and containment.

Referring to FIGS. 1 and 2, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, cellulosic materials, cross-linked cellulosic materials, air laid cellulosic nonwoven materials, spunlace materials, or combinations thereof, for example. In some instances, portions of the acquisition materials 38 may extend through portions of the topsheet 26, portions of the topsheet 26 may extend through portions of the acquisition materials 38, and/or the topsheet 26 may be nested with the acquisition materials 38. Typically, an acquisition material 38 may have a width and length that are smaller than the width and length of the topsheet 26. The acquisition material may be a secondary topsheet in the feminine pad context. The acquisition material may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. Some exemplary surface fastening systems are disclosed in U.S. Pat. Nos. 3,848,594; 4,662,875; 4,846,815; 4,894,060; 4,946,527; 5,151,092; and 5,221,274. An exemplary interlocking fastening system is disclosed in U.S. Pat. No. 6,432,098. The fastening system may also provide a means for holding the article in a disposal configuration as disclosed in U.S. Pat. No. 4,963,140. The fastening system may also include primary and secondary fastening systems, as disclosed in U.S. Pat. No. 4,699,622. The fastening system may be constructed to reduce shifting of overlapped portions or to improve fit as disclosed in U.S. Pat. Nos. 5,242,436; 5,499,978; 5,507,736; and 5,591,152. The ears, or portions thereof, may be elastic or may have elastic panels. In an example, an elastic film or elastic strands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2.

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

“Array” means a display of packages comprising disposable absorbent articles of different article constructions (e.g., different elastomeric materials [compositionally and/or structurally] in the side panels, side flaps and/or belts flaps, different graphic elements, different product structures, fasteners or lack thereof). The packages may have the same brand and/or sub-brand and/or the same trademark registration and/or having been manufactured by or for a common manufacturer and the packages may be available at a common point of sale (e.g. oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

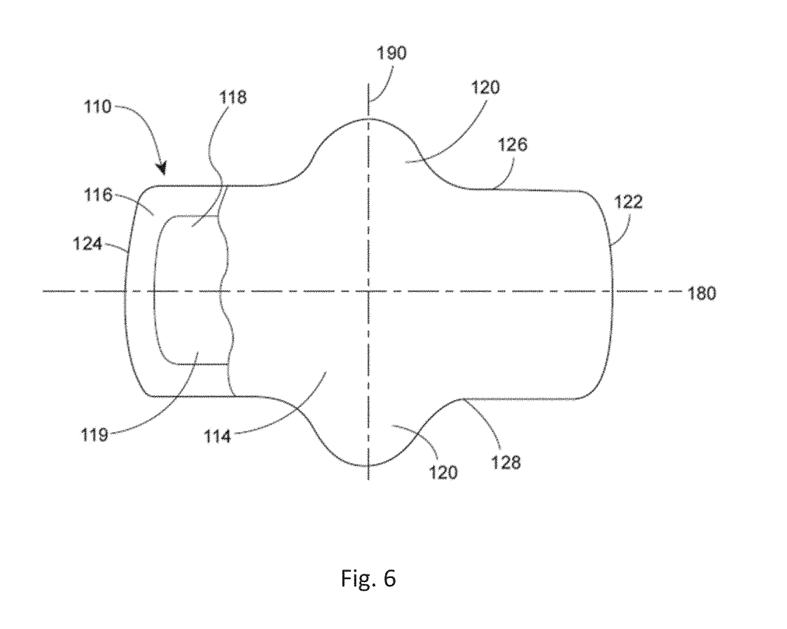

Referring to FIG. 6, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

It is to be appreciated that the features of the absorbent article described herein may be excluded or combined to form various embodiments of an absorbent article.

As previously mentioned, the methods and apparatuses according to the present disclosure may be utilized to assemble discrete absorbent articles 100 and/or various components of absorbent articles 100. Although the following may be provided in the context of absorbent articles 10, as shown in FIGS. 1-6, it is to be appreciated that the methods and apparatuses herein may be used with various process configurations and/or absorbent articles, such as for example, disclosed in U.S. Pat. Nos. 7,569,039 and 9,072,632; U.S. Patent Publication Nos. 2005/0107764 A1, 2012/0061016 A1, and 2012/0061015 A1; 2013/0255861 A1; 2013/0255862 A1; 2013/0255863 A1; 2013/0255864 A1; and 2013/0255865 A1; and U.S. patent application Ser. No. 62/136,003 filed on Mar. 20, 2015 entitled “DISPOSABLE ABSORBENT ARTICLES AND ARRAYS OF SAID ARTICLES COMPRISING VISUAL CHARACTERISTICS”; Ser. No. 14/996,683 filed on Jan. 15, 2016 entitled “ADULT DISPOSABLE ABSORBENT ARTICLES AND ARRAYS OF SAID ARTICLES COMPRISING ABSORBENT CORES HAVING CHANNELS”; and 62/286,662 filed on Jan. 25, 2016 entitled “ABSORBENT ARTICLES COMPRISING SPACERS.”

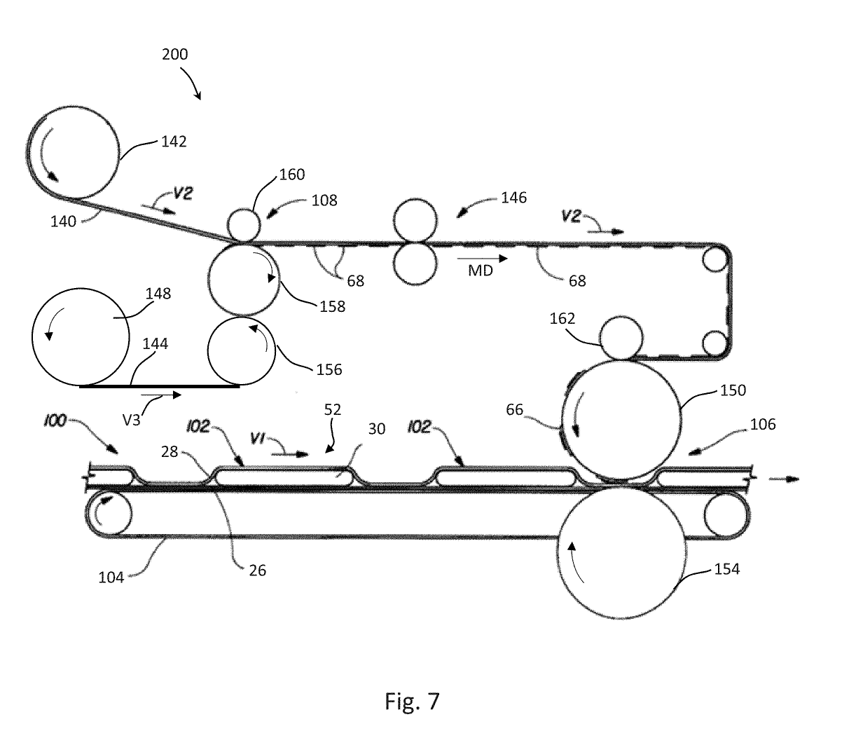

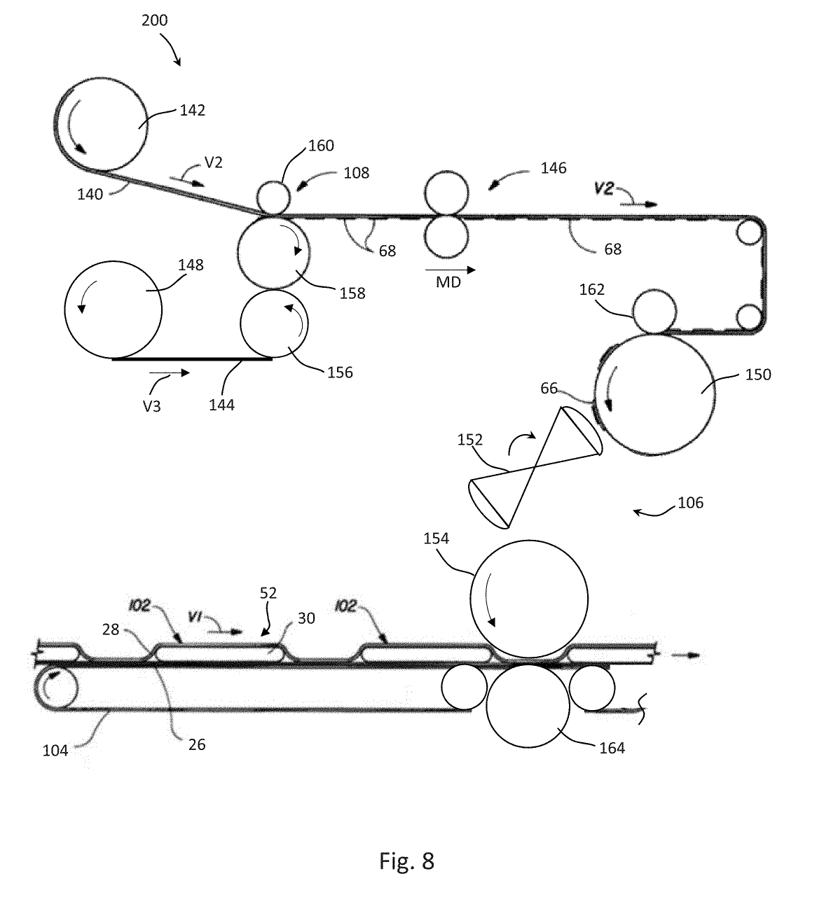

Referring now to FIGS. 7 and 8 there are shown schematic side elevation views each illustrating a method and apparatus for assembling a multi-piece disposable diaper. A continuous web 100 comprised of a plurality of interconnected chassis 102 are fed on conveyer 104 to first assembly station 106. Each individual chassis 52 comprises a liquid pervious topsheet 26, a liquid impervious backsheet 28 and an absorbent core 30. The continuous web 100 comprised of a plurality of interconnected chassis 102 is fed to the first assembly station 106 at a first speed V1. In some embodiments, the first speed V1 may be preferably from about 150 to about 600 meters per minute.

An ear substrate 140 is unwound from supply roll 142 and fed to a second assembly station 108. The ear substrate 140 is fed to the second assembly station 108 at a second speed V2. In some embodiments, for example, the second speed V2 may be less than the first speed V1. The second speed V2 may be from about 100 to about 180 meters per minute. The speed of the ear substrate may be determined with reference to the speed of the continuous web such that the ears (also referred to herein as ear pieces) may be in the desired position for placement on the continuous web. Ear substrate 140 may include a laminate comprising a nonwoven web and a polymeric film. Other suitable materials for the ear substrate 140 include, but are not limited to nonwoven substrates, polymeric films, elastomeric nonwovens, elastomeric films, elastomeric scrim, elastomeric foam, and the like. In the embodiment shown in FIGS. 7 and 8, only a single ear substrate is fed to the second assembly station 108. However, it may be desirable to feed a pair of ear substrates to the second assembly station in order to provide an ear piece on either side of the chassis 52.

A securement member substrate 144 is unwound from supply roll 148 and is fed to the second assembly station 108. The securement member substrate is fed to the second assembly station 108 at a third speed V3. The third speed V3 may be from about 20 to about 60 meters per minute.

The second speed V2 may be different than the third speed V3. Similarly, the second speed may be different than the first speed V1, and the third speed V3 also may be different than the first speed V1. For example, in some embodiments, the second speed V2 (the speed of the ear substrate), may be less than the first speed V1 (the speed of the chassis) and that the third speed V3 (the speed of the securement member substrate) may be less than the second speed V2. This enables the securement member substrate to be cut into individual securement members and secured to the ear substrate in the appropriate position and then allow the ear substrate to be cut into individual ear pieces 66 and secured to the chassis in the appropriate position.

At the second assembly station 108, the securement member substrate 144 is cut into individual securement members 68 which are then bonded to the ear substrate 140. The securement member substrate 144 may comprise any securement member substrate known in the art. Examples of suitable securement substrates include adhesive tape substrates, mechanical fastener substrates, combination mechanical and adhesive fastener substrates, and the like. It is also to be appreciated that the securement member substrate may include two or more different materials. For example, the securement member substrate may comprise a nonwoven substrate and a portion of the substrate may include a fastener 46, such as adhesive and/or a mechanical fastener, hooks/loops. It is also to be appreciated that the entire substrate may be made of the material that is used for fastening. For example, the entire substrate may include adhesive and/or mechanical fasteners, such as hooks/loops.

After the securement member substrate 144 has been cut into individual securement members 68 such as individual adhesive tape tabs, the individual adhesive tape tabs may be bonded to the ear substrate using any suitable method. Examples of suitable methods for bonding the individual securement members to the ear substrate include but are not limited to adhesive bonding, cohesive bonding, ultrasonic bonding, heat bonding, pressure bonding, friction bonding, autogenous bonding or combinations of bonding methods. In some embodiments, a pair of securement member substrates are fed to the second assembly station 108 and are cut into individual securement members 68 and secured to either side of the ear substrate 140. The ear substrate having the individual securement members bonded thereto is then slit into two ear substrates at slitter 146 to provide an ear 66 on either side of the chassis.

Referring again to FIG. 1, the absorbent article 10 is shown to comprise a pair of securement members 68, such as hook/loop tabs secured to the individual ear pieces 66, also referred to herein as ears. The securement members 68 may each comprise a fixed end 82 and a refastenable end 84. The fixed end 82 is the portion that is bonded to the ear 66 during manufacture. The refastenable end 84 is the portion that extends laterally outwardly beyond the edge of the ear 66 and that may be grasped by the diaperer in securing the diaper on the wearer. The fixed end may be secured to either the outer surface of the ear piece or the inner surface of the ear piece. The fixed end may also be secured between layers of the ear where the ear includes a multi-layer structure.

Referring now to FIGS. 7 and 8, the ear substrates including the individual securement members bonded thereto may be then fed to the first assembly station 106. The ear substrates including the securement members may be fed at the second speed V2. At the first assembly station 106 the ear substrate including the securement members bonded thereto may be cut into individual ear pieces 66. The individual ear pieces 66 are then bonded to the chassis at the first assembly station 106 to form multi-piece absorbent articles. The individual ear pieces 66 may be bonded to the chassis using any suitable method. Examples of suitable methods for bonding the individual ears 66 to the chassis include but are not limited to adhesive bonding, cohesive bonding, ultrasonic bonding, heat bonding, pressure bonding, friction bonding, autogenous bonding or combinations of bonding methods. The individual ear pieces 66 may be bonded to the backsheet, the topsheet or both. The individual ear pieces 30 may also be inserted between the topsheet and backsheet and bonded to both. In an example embodiment, the individual ear pieces 66 may be bonded directly to the backsheet. The continuous web 100 of interconnected chassis 102 having the ears bonded thereto may be then cut into individual multi-piece disposable diapers. The cutting may take place at the first assembly station 106.

In some embodiments, as illustrated in FIG. 7, the first assembly station 106 may include a first cutting roll 150 configured to separate a substrate generally in the cross direction MD, which is substantially perpendicular to the machine direction MD. It is also to be appreciated the first cutting roll 150 may include a device which cuts a pattern in the substrate, thus, cutting the substrate in multiple directions, which may include both the cross direction CD and the machine direction MD.

The first cutting roll 150 may be positioned adjacent a first anvil roll 162. The first anvil roll may operatively engage the cutting roll to aid in cutting the substrate. The first cutting roll may also be positioned adjacent a first bonding roll 154. The first bonding roll 154 may operatively engage a portion of the cutting roll 150 causing a portion of the chassis to engage the ear pieces 66 and bonding the ear pieces 66 to a portion of the chassis. As previously discussed, the ear pieces 66 may be bonded to the chassis in various ways.

In some embodiments, as illustrated in FIG. 8, the first assembly station 106 may include a first cutting roll 150 configured to separate a substrate generally in the cross direction MD, which is substantially perpendicular to the machine direction MD. It is also to be appreciated the first cutting roll 150 may include a device which cuts a pattern in the substrate, thus, cutting the substrate in multiple directions. As previously discussed, the first cutting roll 150 may be positioned adjacent a first anvil roll 162, which may operatively engage the cutting roll to aid in cutting the substrate, and a transfer device 152. The first cutting roll 150 may cut and advance the ear piece toward the transfer device 152. The ear may be transferred from the first cutting roll 150 to the transfer device 152. The transfer device 152 may be used to accelerate and/or decelerate a portion of the substrate, such as the ear piece, such that the ear piece advances at the speed of the chassis or substrate onto which the ear piece is to be deposited. The transfer device 152 may be a device such as described in U.S. Pat. Nos. 6,450,321; 6,705,453; 6,811,019; and 6,814,217. The transfer device 152 may deposit the individual ear pieces onto a first bonding roll 154 positioned adjacent the transfer device 152. The first bonding roll 154 may be used to bond the individual ears to the interconnected chassis 102 and/or the chassis 52. The first bonding roll 154 may also be positioned adjacent a second anvil roll 164. The anvil roll 164 may operatively engage the first bonding roll 154 such that the anvil roll 164 the interaction causes the ear piece to be deposited and bonded to the chassis.

Referring to FIGS. 7 and 8, in some embodiments, the second assembly station 108 may include a second cutting roll 156. The second cutting roll 156 may be positioned downstream, in the machine direction MD of the supply roll 148. The second cutting roll 156 may be used to cut the securement member substrate 144. The securement member substrate 144 wraps around a portion of the second cutting roll 156 and cuts the securement member substrate 144 into securement members 68. The individual securement members 68 may be transferred to a transfer roll or anvil roll 158. The transfer roll/anvil roll 158 may be configured to operatively engage a second bond roll 160. The anvil roll 158 and second bond roll 160 cause the securement member 68 to engage and bond to the ear substrate 140. It is to be appreciated that in some embodiments, the cutting and bonding may be done with a single assembly station. For example, the second cutting roll 156 may be used to cut the securement member substrate 144 into securement members 68 and may also be used to bond the securement members 68 to the ear substrate 140.

As the one or more substrates and portions thereof advance through the process and apparatus described above, alterations made to the substrate and placement of the invidious portions of the substrate need to be performed with accuracy and precision. The use of separate registration marks, such as lines printed on substrate in specific areas or with specific properties, such as being identifiable by specific type of light, has been used to identify portions of the substrate or individual pieces thereof for processing. Further, substrates have been designed such that there are specific portions that are devoid of graphics or other aesthetic features of the absorbent article so that again portions of the substrate or individual pieces thereof could be identified for processing. The intentional lack of aesthetic features may be both noticeable and displeasing to the consumer. The present disclosure addressed this problem by using a sensor that is able to be taught registration features based on the graphics or other aesthetic or function features of the absorbent article. Thus, there is no need to add extra registration marks or to leave portion of the article devoid of features.

As previously discussed, the securement member substrate 144 and the ear substrate 140 may include a plurality of features. These features 90 may include graphics, bonds, visually discernible patterns of three-dimensional features, or any other contrasting area, such as notches, protrusions, and depressions, for example. The graphics may be printed, impregnated, and/or coated on the substrate as is known in the art and such as described, for example, in U.S. Pat. Nos. 9,226,857; 9,408,753; and 9,724,244. The bonds may include a pattern of individual bonds and/or one or more continuous bonds, such as a bond that depicts some sort of object or item, which may be, for example, a bear and/or a balloon. Further, the visually discernible patterns of three-dimensional features may include zones having various properties based on the method and/or apparatus for manufacturing the substrate such as described in U.S. Publication Nos. 2017/0027774 A1; 2017/0029993 A1; 2017/0191198 A1; and 2017/0029994 A1 and U.S. Application Nos. 62/527,216; 62/527,224; 62/452,518; 62/452,566; and 62/434,547.

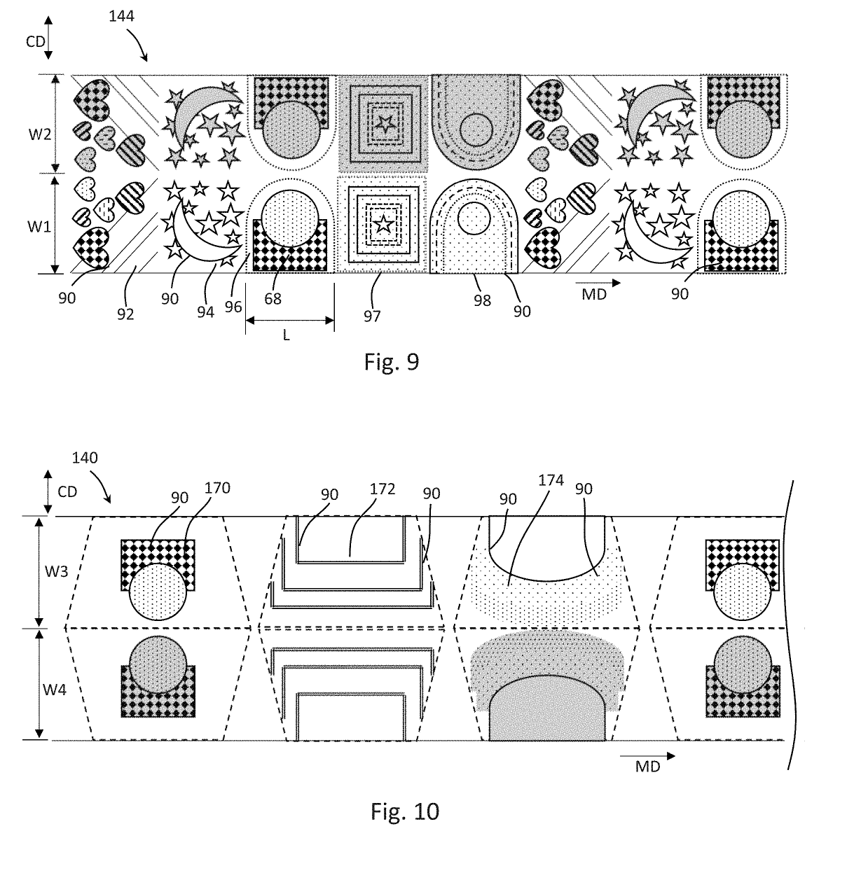

The features 90 may cover a substantial portion of the substrate in the machine direction MD. As previously discussed, the sensor is able to discern portions of the features, referred to herein as registration features, so that separate, independent registration marks or areas that are intentionally left blank, featureless, are not needed. Thus, a substantial portion of the substrate may be covered by features 90 in the machine direction MD. In some embodiments, for the length L of a given element pitch, which may be the length in the machine direction MD from the beginning of one element to the end of that element, extending in the machine direction MD, at least about 75% and/or at least about 80% and/or at least about 85% and/or at least about 90% and/or at least about 98% of the element includes one or more features. For example, as illustrated in FIG. 9, a securement member substrate 144 comprises features that define what will be a securement member 68, such as illustrated in FIGS. 12A and 12B, once the securement member substrate 144 undergoes cutting. Each securement member 68 has a length L that is parallel to the machine direction MD. The length L would be the element pitch for the securement member 68, which is the element. As illustrated in FIG. 9, the securement member 68 includes various features that extend over a substantial portion of the securement member 68 in the machine direction MD. It is to be appreciated that the coverage of the features in the machine direction MD may be applicable to various types of substrates, such as the ear substrate, a backsheet substrate, a topsheet substrate, and a landing zone substrate.

The securement member substrate 144 may comprise a plurality of features 90 that define what will be the securement members 68 once the securement member substrate 144 is cut. For example, as illustrated in FIG. 9, a first group of features define a first pattern 92. The first pattern 92 may be adjacent to a second group of features that define a second pattern 94, which may be the same as or different than the first pattern 92. Further, the second pattern 94 may be adjacent to a third group of features that define a third pattern 96, which may be the same as or different than each of the first pattern 92 and the second pattern 94. Similarly, a fourth group of features may define a fourth pattern 97 and a fifth group of features may define a fifth pattern 98. The fourth pattern 97 may be positioned between the third pattern 96 and the fifth pattern 98. Each of the aforementioned patterns may be different or two or more patterns may be the same. The securement member substrate 144 may include repeating patterns such as illustrated in FIG. 9. Thus, a certain number of patterns may be presented in a specific order and this order may repeat once each unique pattern has been presented. It is to be appreciated that each of the patterns on the substrate may be unique and, thus, no repeat pattern is present. It is also to be appreciated that each of the patterns may be the same.

The securement member substrate 144 may also include a first pattern width W1, extending in the cross direction CD, and a second pattern width W2, also extending in the cross direction CD. The first pattern width W1 may include a first series of patterns such as illustrated in FIG. 9. The second pattern width W2 may include a mirror image of the first series of patterns such as illustrated in FIG. 9. The first pattern width W1 and the second pattern width W2 include mirror images of one another such that when the securement member substrate 144 is cut, matching securement members 68 may be present on each side of the chassis of the absorbent article. It is to be appreciated that the first pattern width W1 and the second pattern width W2 do not need to be mirror images of one another. For example, one securement member may include a different feature than the opposing securement member on the absorbent article. The first securement member may include characters “A, B, C” and the opposing, second securement member may include characters “E, F, G.”

The ear substrate 140 may comprise a plurality of features 90 that may be present on the ear pieces 66 once the ear substrate 140 is cut. For example, as illustrated in FIG. 10, a first group of features define a first ear pattern 170. The first ear pattern 170 may be adjacent to a second group of features that define a second ear pattern 172, which may be the same as or different than the first pattern 92. Further, the second ear pattern 172 may be adjacent to a third group of features that define a third ear pattern 174, which may be the same as or different than each of the first ear pattern 170 and the second ear pattern 172. Each of the aforementioned patterns may be different or two or more patterns may be the same. The ear substrate 140 may include repeating patterns such as illustrated in FIG. 10. Thus, a certain number of patterns may be presented in a specific order and this order may repeat once each unique pattern has been presented. It is to be appreciated that each of the patterns on the substrate may be unique and, thus, no repeat pattern is present. It is also to be appreciated that each of the patterns may be the same.

The ear member substrate 140 may also include a first ear pattern width W3, extending in the cross direction CD, and a second ear pattern width W4, also extending in the cross direction CD. The first ear pattern width W3 may include a first series of patterns such as illustrated in FIG. 10. The second ear pattern width W4 may include a mirror image of the first series of patterns such as illustrated in FIG. 10. The first ear pattern width W3 and the second ear pattern width W4 may include mirror images of one another such that when the ear substrate 140 is cut, matching ears 66 may be present on each side of the chassis of the absorbent article. It is to be appreciated that the first ear pattern width W3 and the second ear pattern width W4 do not need to be mirror images of one another. For example, ear 66 may include a different feature than the opposing ear 66 on the absorbent article. The first ear may include characters “A, B, C” and the opposing, ear securement member may include characters “E, F, G.”

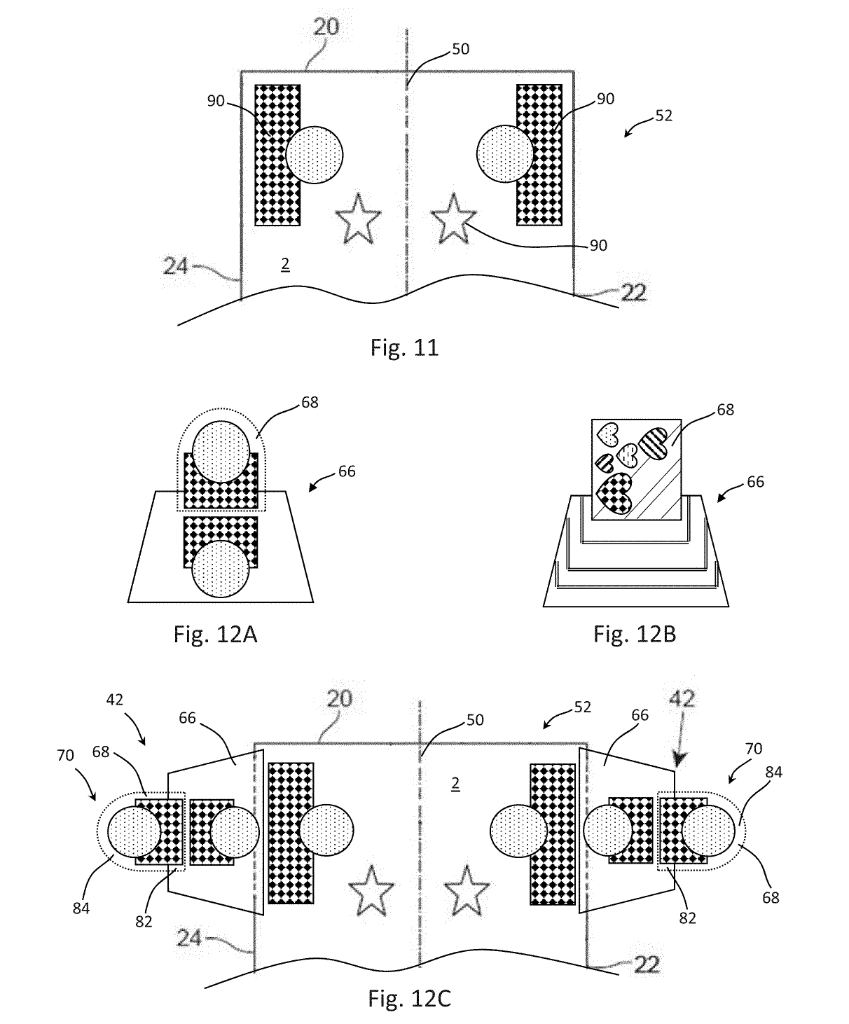

The chassis 52 may also comprise a plurality of features 90. These features 90 may coordinate with the features present on the securement member 68 and/or the ear 66. It is also to be appreciated that the features 90 included in the chassis 52 may be different than the features included in the securement member 68 and/or the ear 66. The features 90 included on the chassis 52 may be positioned such that the ear 66 is positioned with respect to the features 90. For example, as illustrated in FIG. 11, the chassis 52 may include a plurality of features 90. These features 90 may coordinate with the ear 66 and the securement member 68, as illustrated in FIG. 12A. The chassis 90 may also include other features 90, shown as stars in FIG. 11, that do not necessarily coordinate with the ear 66 and/or the securement member 68. The features 90 present on the chassis 52 may indicate where the ear 66 and securement member 68 are to be placed and bonded.

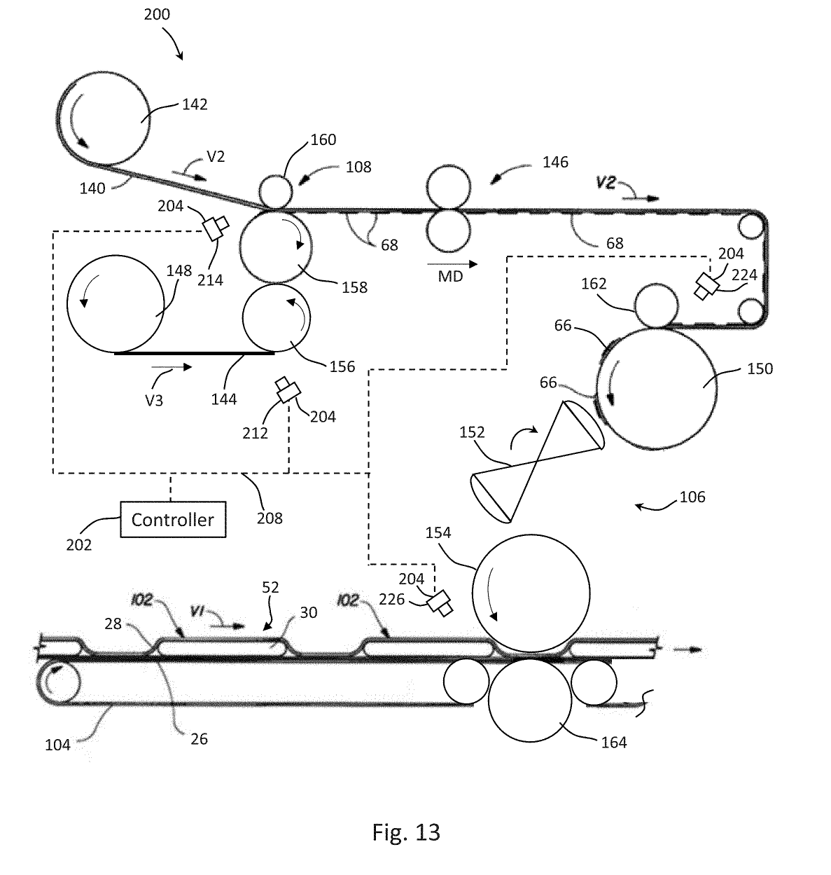

As discussed in more detail below, converting apparatus 200, also referred to herein as the converting line, may control the manufacture and relative placement of components of absorbent articles, such as the securement members 68 and the ear pieces 66 during the assembly process. For example, in some configurations, the relative placement of the securement members 68 and/or the ears 66 may be controlled to align the features present on the securement members 68 and the ears 66 in a desired manner. For example, as shown in FIG. 13, the converting apparatus may include a controller 202 adapted to change the machine direction speeds and/or cross direction positions of advancing substrates or portions thereof, such as elastic laminates and/or chassis. Such changes in machine direction speeds and/or cross direction positions may be based on the detection of registration features. In turn, the changes in machine direction speeds and/or cross direction positions may alter the relative alignment of the features present on each of the components, such as securement members 68, ears 66, and/or chassis 102.

An inspection system, described as follows, may be configured to interact with, monitor, and/or control the converting apparatus 200. As shown in FIG. 13 and as described in more detail below, various sensors 204 and other devices may be arranged adjacent the converting line 200 and may communicate with a controller 202. Based on such communications, the controller 202 may monitor and affect various operations on the converting line 200. For example, the controller may send various types of control commands to the converting line, such as speed change commands based on communications with the sensors 204. In some embodiments, the control commands may be in the form of reject commands. In the systems and methods described herein, the controller 202 may include one or more computer systems. The computer system may, for example, include one or more types of programmable logic controller (PLC), programmable automation controller (PAC) and/or personal computer (PC) running software and adapted to communicate over an industrial network. Some embodiments may utilize industrial programmable controllers such as the Siemens S7 series, Rockwell ControlLogix, SLC or PLC 5 series, or Mitsubishi Q series and industrial networks such as Profinet, Modbus TCP, Ethernet/IP, and CC_Link. The aforementioned embodiments may use a personal computer or server running a control algorithm such as Rockwell SoftLogix or National Instruments Labview or may be any other device capable of receiving inputs from sensors, performing calculations based on such inputs and generating control actions through servomotor controls, electrical actuators or electro-pneumatic, electrohydraulic, and other actuators. Process and product data may be stored directly in the controller or may be located in a separate data historian. In some embodiments, the historian is a simple data table in the controller, in other embodiments, the historian may be a relational or simple database. Common historian applications include Rockwell Automation Factory Talk Historian, General Electric Proficy Historian, OSI PI, or any custom historian that may be configured from Oracle, SQL or any of a number of database applications.

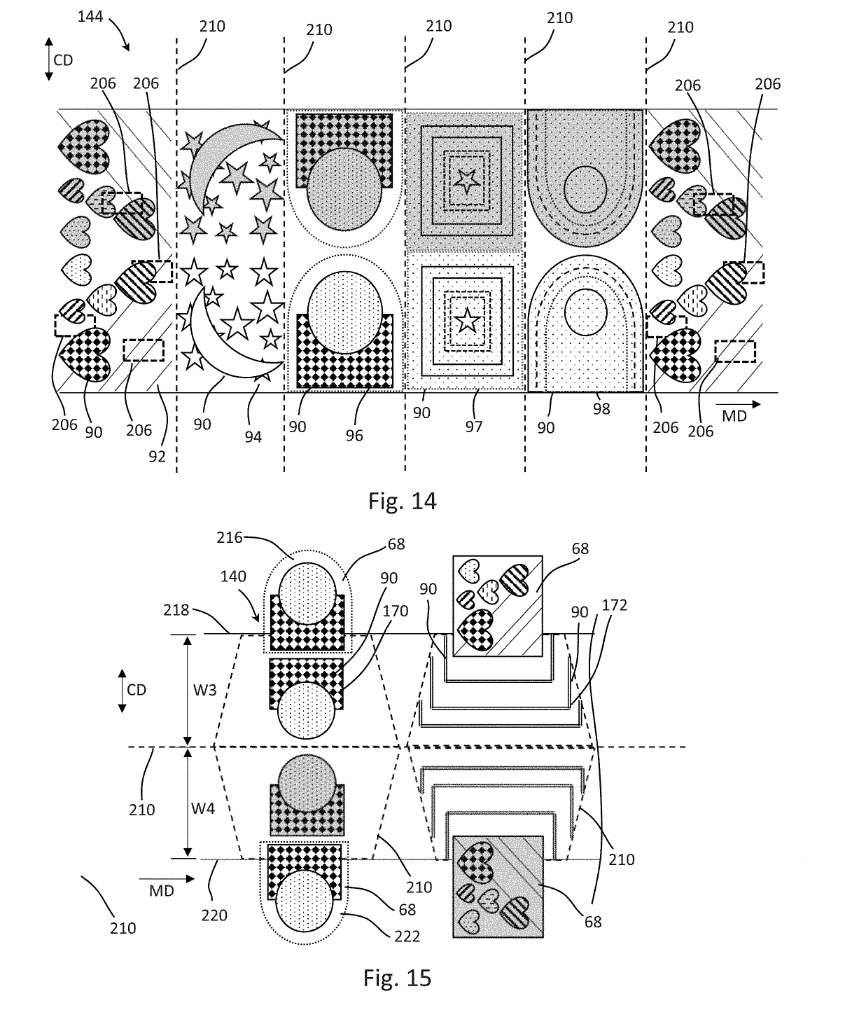

As previously described with reference to FIGS. 9 and 10, the securement members 68, the ears 66, and/or the continuous length of chassis assemblies 102 may include features. These features may be used by the sensor to identify registration features 206, such as illustrated in FIG. 14. A registration feature 206 may be any area of contrast that may be repeatedly recognized by a mechanical device, such as a sensor. For example, as illustrated in FIG. 14, the securement member substrate 144 may include a plurality of features. The sensor may be taught to identify registration features 206 present on the first pattern 92. The registration features 206 may be any feature and/or portion of a feature. The sensor may identify several registration features for a given pattern. As illustrated in FIG. 14, the sensor identifies four registration features on the first pattern 92. The sensor generates an output each time it recognizes the registration features present on the first pattern 92. It is to be appreciated that the sensor may identify registration feature across patterns. For example, the sensor may identify registration features on the first pattern 92 and the second pattern 94. It is also to be appreciated that the sensor may identify any number of registration features to enable continued recognition of the pattern. For example, the sensor may identify at least two registration features and/or at least four registration features and/or at least about six registration features and/or at least about eight registration features and/or at least about ten registration features.

The sensor 204 may be taught a first pattern based on first registration features that are particular to the first pattern. The sensor 204 may then use those first registration features to identify the pattern and generate a signal. The sensor 204 may also be taught a second pattern based on a different, second registration features that are particular to the second pattern. The sensor 204 may then use those second registration features to identify the second pattern and generate a signal. The first registration features may be stored in memory so that they may be recalled to the sensor at any time. Similarly, the second registration features may be stored in memory so they may be recalled to the sensor at any time. Thus, the sensor may toggle between learned registration features. Thus, any number of different sets of registration features that have been stored may be recalled. This function may be valuable when manufacturers switch from manufacturing products with different features. It is also to be appreciated that each sensor present in the converting line 200 may be taught to detect the same registration features or different registration features.

The sensor may use light to aid in identifying the registration features and the sensor may use filters to limit the amount or frequency of light received. This light may be white, ultraviolet, infrared, or polarized, and filter types may include low-pass, high pass, band pass, band-stop, and polarizing. The light or filter may be chosen based on the particular registration features. The type of light or filter may optimize the contrast of the features. For example, white light may be used for features which are graphics.

The sensor may also account for a certain amount of stretch in the substrate. During processing, the substrate may undergo stretching due to tensions from the various process equipment. The sensor may account for that stretch and still recognize the pattern using the taught registration features despite some elongation and/or narrowing in the substrate.

The sensor may also be able to identify the patterns based on the registration features at relatively high speeds. For example, the sensor may identify the registration features as the substrate advances at a speed of from about 0.1 m/s to about 10 m/s. It is to be appreciated that higher speeds may be possible with a corresponding reduction in detection accuracy or increase in sensor data acquisition rate and computational power.

The systems and methods herein may utilize various types of sensors to monitor the substrates and components traveling through the converting line 300. As shown in FIG. 13, various types of sensors 204 may be used to perform various functions. For example, sensors 204 may be used to detect registration features 206, the relative placement of substrates and/or components, and various types of defects. Based on the detections of the sensors 204, feedback signals from the sensors may be communicated to the controller 202. For example, a sensor 204 can be configured to detect a registration feature 206 on a substrate and communicate an inspection parameter corresponding with the detection of the registration feature to the controller 202. The controller 202 receives the feedback signals from the sensors, associates a time-stamp or position with the signal, and compares the detected position or time of the feedback signal with ideal positions or times determined by the process setpoints. Based on the comparison, the controller may change the machine direction speeds and/or cross directional positions of the substrates. It is also to be appreciated that various types of controllers and sensors can be configured in various ways and with various algorithms to provide various types of data and perform various functions, for example, such as disclosed in U.S. Pat. Nos. 5,286,543; 5,359,525; 6,801,828; 6,820,022; 7,123,981; 8,145,343; 8,145,338; 8,145,344; 8,244,393; 8,712,573; 8,712,574; and 9,429,929; and European Patent No. EP 1 528 907 B1, all of which are incorporated by reference herein.

It is to be appreciated that various different types of inspection sensors 602 may be used to detect registration features and monitor the substrates and components while advancing through the converting line 300. For example, sensors 204 may be configured as photo-optic sensors that receive either reflected or transmitted light and serve to determine the presence or absence of a specific material; metal-proximity sensors that use electromagnetic to determine the presence or absence of a ferromagnetic material; capacitive or other proximity sensors using any of a number of varied technologies to determine the presence or absence materials. Sensors 204 may also be configured as vision systems and other sub-processing devices to perform detection and, in some cases, logic to more accurately determine the status of an inspected product.

Particular examples of inspections sensors 204 may include a pattern sensor such as the SICK PS30 provided by SICK, Inc. The PS30 sensor is a pattern scanning opto-electronic sensor. The sensor may be taught distinctive areas of a pattern or patterns that are used as a reference for subsequent reliable detection and positioning of objects. Another example of a sensor 204 may include a full-spectrum sensor such as the LR-W Series available from Keyence Corporation. The LR-W Series sensor may detect differences based on appearance through color and/or contrast. Additional examples of sensors 204 may include the Cognex Checker 4G or Cognex is2000. These series can perform feature detection and output a signal based on detected position. In another example, inspection sensors 204 may include combinations of commercial components in a system such as a GigE or USB 3.0 camera available from Basler, an industrial processor with FPGA discrete I/O and/or Ethernet communication capability, such as National Instruments' (NI) Industrial Controller IC-31XX, and software with pattern detection capability such as NI's Vision Builder for Automated Inspection (VBAI).

As shown in FIG. 13, the inspection sensors 204 are connected with the controller 202 through a communication network 208, which allows the sensors 204 to communicate inspection parameters to the controller 202. It should also be appreciated that the inspection parameters may be provided from inspection sensors 204 in various forms. In one embodiment, inspection parameters may be in the form “results,” such as for example, provided from a sensor state change resulting in a binary input corresponding with the detected presence or absence of a registration feature. This binary input change may be detected using an input-output (I/O) device or servo drive input resulting in a detection time or position being sent to the controller 202 over the communication network 208. In some embodiments, inspection parameters may be provided in the form of measurements and/or numerical indications of detected positions of registration features relative to components and/or substrates; and/or numerical indications of the positions of components and/or substrates relative to other physical, virtual, or time references. In other embodiments, inspection parameters may be in the form of images transferred via a standard protocol such as ftp (File Transfer Protocol), DDE (Dynamic Data Exchange), or OPC (Object Linking and Embedding for Process Control).

As mentioned above, the controller 202 may be adapted to control the relative placement of the securement member substrate 144, the ear substrate 140, the securement members 68, the ears 66 and/or chassis 102 during the assembly process based on detections of one or more registration features 206. For the purposes of the following discussion, with reference to FIG. 13, the continuous web 100 of interconnected chassis 102 may advance in the machine direction MD at a speed V1; and the ear substrate 140 may advance in the machine direction MD at a speed V2; and securement member web 144 may advance in the machine direction MD at a speed V3.

The controller 202 may be configured to change the placement of the registration features relative to each other and/or to other registration features along the machine direction MD by adjusting the speed of the substrates and/or individual members/pieces and/or shifting the substrates and/or individual members/pieces in the cross direction CD. As illustrated in FIG. 13, the securement member substrate 144 advances from a supply roll 142 to a second cutting roll 156. A first sensor 212, 204 may be used to detect first registration features 206 of the securement member substrate 144. As illustrated in FIGS. 9 and 14, the first sensor 212, 204 may be taught to recognize the first registration features 206 based on the features of the securement member substrate 144. The sensor 204 may communicate with the controller 202 such that the securement member substrate 144 undergoes cutting in the desired location based on the first registration features 206. For example, the securement member substrate 144 may be cut along cut lines 210, as illustrated in FIG. 14, to form individual securement members 68. It is to be appreciated that the communication between the sensor 204, the controller 202, and the process equipment enables the securement member substrate 144 to be cut in the desired location. The securement member substrate 144 may be adjusted in the machine direction MD, which may be an increase or decrease in speed, and in the cross direction CD, such that the substrate is appropriately aligned.

The securement member substrate 144 may advance to a transfer roll 158. As described earlier this roll 158 may also function as an anvil roll to aid in cutting the securement member substrate 144. The transfer roll 158 may be used to advance the securement members 68 such that each securement member may be placed on the ear substrate 140 in a desired location. The ear substrate 140 may traverse at a second speed which may be different than the third speed at which the securement member substrate 144 traverses. The difference in speed may aid in proper placement of the securement member 68 on the ear substrate 140. For example, the third speed may be less than the second speed so that the securement members 68 are spaced apart in the machine direction MD when applied to the ear substrate 140.

Further, the ear substrate 140 may include one or more features, such as previously described. A second sensor 214, 204 may be used to identify second registration features 206 on the ear substrate 140. The second sensor 214, 204 may be taught to identify the second registration features 206 based on the features of the ear substrate 140. The identification of the second registration features 206 may aid in controlling the ear substrate 140 and/or the transfer roll 158 such that the securement member 68 is placed in the desired location on the ear substrate 140. For example, as illustrated in FIG. 15, the securement member 68 may be placed on the ear substrate 140 such that the features of the ear substrate 140 coordinate with the features of the securement member 68. More specifically, a first securement member 216 may be positioned adjacent a first side edge 218 of the ear substrate 140 such that a portion of the first securement member 216 extends beyond the first side edge 218 in a direction parallel to the cross direction. It is to be appreciated that the securement member does not have to extend beyond the first side 218 of the ear substrate 140. The securement member may be attached to the ear substrate 140 in a folded orientation or may be attached and later folded. Similarly, a second securement member 222 may be positioned and attached adjacent a second side edge 220 of the ear substrate 140. The first securement member 216 may be the same as or different than the second securement member 222. As the ear substrate 140 advances in the machine direction MD the securement members 68 are disposed on and bonded to the ear substrate 140. A second bond roll 160 bonds the securement members 68 to the ear substrate 140.

In some embodiments, the ear substrate 140 including the securement members 68 bonded thereto advances to a slitter 146. The slitter 146 slits the ear substrate 140 including the securement members 68 in a direction substantially parallel to the machine direction MD. For example, FIG. 15 illustrates a cut line 210 along which the slitter 146 may cut the ear substrate 140.

The slit ear substrate 140 including the securement members 68 advances to a first cutting roll 150 and an anvil roll 162. A third sensor 224 may be used to detect third registration features based on the features of the ear substrate 140 including the securement members 68. The third registration features may be the same as or different than the first registration features and the second registration features. Based on the location of the third registration features, each of the slit ear substrates 140 including the securement members 68 may be cut into individual ear pieces 66, also referred to as ears 66. The third sensor 224 may signal the controlled 202 to adjust, for example, the speed of the ear substrates 140 and/or the speed of the first cutting roll 150. FIG. 15 illustrates an example of the cut lines 210 along which the ear substrate 140 may be cut to form the ears 66. The ear substrate 140 may be cut in any desired shape such as rectangular, trapezoidal, peanut-shaped, or any other shape. It is to be appreciated that the ear substrate 140 may be cut into any number of shapes. For example, a first ear may be cut into a trapezoidal shape and a second ear may be cut into a rectangular shape. An example of ears 66 which have been cut from an ear substrate 140 are illustrated in FIGS. 12A and 12B.

It is to be appreciated that a third and fourth sensor may be used to detect registration features on each of the slit ear substrates. More specifically, the ear substrate may be split into a first portion and a second portion. The third sensor may be used to detect registration features on the first portion of the slit ear substrate and the fourth sensor may be used to detect registration on the second portion of the slit ear substrate.

Referring to FIG. 13, the ears 66 may be advanced to the transfer device 152, such as previously described. The transfer device 152 is configured to receive the ear 66 from the first cutting roll 150. The transfer device 152 may then accelerate and/or decelerate the ears 66 such that the ears may be transferred to a first bonding roll 154 that rotates at the first speed V1. The transfer device 152 may also provide the approximate spacing of the ears 66 such that each ear is placed in a desired location on the chassis 102. The transfer device 152 may use vacuum to aid in transferring, advancing, and depositing the ears 66. The transfer device 152 may deposit the ear 66 onto the first bonding roll 154. The first bonding roll 154 may operatively engage the second anvil roll 164 to bond the ears 66 to the chassis. A fourth sensor 226 may be used to identify fourth registration features based on the features of the chassis. As illustrated in FIG. 11, the chassis 52 may include one or more features. A portion of these features may correspond to the ear 66 and/or the securement member 68. The fourth sensor 226 may be taught to identify fourth registration features based on the features of the chassis. The ear 66 may then be bonded to the chassis based on the fourth registration features. The fourth sensor 226 may signal the controller 202 to adjust the speed of the bonding roll 154, for example. As illustrated in FIG. 12C, the ear 66 is placed with respect to a feature of the chassis 52. Each chassis may have the same features or different features. Once the ears 66 have been bonded to the chassis, the chassis including the ears 66 may advance to other downstream processes such as additional cutting process, folding processes, and/or additional bonding processes.

It is also to be appreciated that the sensor may communicate with the controller 202 to indicate a need for change in the cross direction and such a change may be made. The substrates and/or the individual members/pieces may be shifted in the cross direction based on feedback from the sensors.

It is also to be appreciated that the sensors described above may be used in other processes, such as the process to from a pant-type disposable absorbent article, such as described with reference to FIGS. 4 and 5, and a sanitary napkin, as described with reference to FIG. 6. For example, the second belt 56; the first belt 54; and the continuous length of chassis assemblies may all include features. One or more sensors may be placed along the manufacturing process to identify registration features based on the features. The registration features may be used to cut, bond, and transfer the first belt, second belt, topsheet, core channels (such as described in U.S. Pat. No. 8,979,815 and U.S. Publication Nos. 2012/0316526 A1 and 2013/0327941 A1), backsheet, the chassis and/or any other discrete portion of the absorbent article. For example, as previously described, the registration features may include bond patterns, graphics, or structural features present on the aforementioned. In another example, the chassis and the landing zone may each include one or more features. The sensor may be used to cut the continuous substrate of landing zones and position and bond the landing zone on the chassis such that the features of each component are in the desired location.

This application claims the benefit of U.S. Provisional Application No. 62/551,932, filed on Aug. 30, 2017, the entirety of which is incorporated by reference herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for assembling a multi-piece absorbent article, the method comprising:
feeding a chassis to a first assembly station at a first velocity, the chassis comprising a topsheet, a backsheet joined to the topsheet, and an absorbent core positioned between the topsheet and the backsheet;
feeding an ear substrate to a second assembly station at a second velocity, wherein the second velocity is different than the first velocity;
feeding a securement member substrate to the second assembly station at a third velocity, wherein the third velocity is different than the second velocity,
wherein the securement member substrate comprises first registration features and the ear web comprises second registration features;
identifying the first registration features using a first sensor;
cutting the securement member substrate into individual securement members based on a position of the first registration features;
identifying the second registration features using a second sensor;
bonding the individual securement members to the ear substrate at the second assembly station, wherein each of the individual securement members is positioned with respect to an individual feature on the ear substrate;
cutting the ear substrate into individual ear pieces; and
bonding the individual ear pieces to the chassis at the first assembly station to form a multi-piece absorbent article.

2. The method of claim 1, wherein the ear substrate comprising the individual securement members comprises a third registration feature.

3. The method of claim 2, comprising identifying the third registration feature using a third sensor, and wherein the ear substrate is cut based on a position of the third registration feature.

4. The method of claim 3, comprising transferring the individual ear pieces, wherein each ear piece is at least one of accelerated and decelerated such that the ear piece advances at the first velocity.

5. The method of claim 1, wherein the second velocity is less than the first velocity, and wherein the third velocity is less than the second velocity.

6. The method of claim 1, wherein each of the first registration feature and the second registration feature comprises at least a portion of at least one of a graphic, a bond, and a visually discernible pattern of three-dimensional features.

7. The method of claim 1, comprising training the first sensor based on the first registration feature.

8. The method of claim 1, comprising training the second sensor based on the second registration feature.

9. The method of claim 1, further comprising slitting the ear substrate to form a pair of ear substrate and feeding the pair of ear substrates to the first assembly station

10. The method of claim 1, wherein the ear substrate comprises a laminate.

11. The method of claim 1, wherein the individual securement members have a fixed end and a refastenable end, and wherein the fixed end of the individual securement members are bonded to the ear substrate.

12. The method of claim 1, wherein the individual ear pieces are bonded to at least one of the backsheet and the topsheet.

13. The method of claim 1, wherein the individual ear pieces are bonded between the topsheet and the backsheet.

14. The method of claim 1, wherein bonding the individual securement members to the ear substrate and bonding the individual ear pieces to the chassis comprises a bonding method selected from the group consisting of adhesive bonding, cohesive bonding, ultrasonic bonding, heat bonding, pressure bonding, friction bonding, autogenous bonding or combinations thereof.

15. A method for assembling a multi-piece absorbent article, the method comprising:
feeding a chassis to a first assembly station at a first velocity, the chassis comprising a topsheet, a backsheet joined to the topsheet, and an absorbent core positioned between the topsheet and the backsheet;
feeding an ear substrate to a second assembly station at a second velocity;
feeding a securement member substrate to the second assembly station at a third velocity,
wherein the securement member substrate comprises first registration features and the ear substrate comprises second registration features;
detecting positions of the first registration features using a first sensor;
cutting the securement member substrate into individual securement members based on the position of each of the first registration features;
detecting positions of the second registration features using a second sensor;
bonding the individual securement members to the ear substrate at the second assembly station based on the position of each of the second registration features;
feeding the ear substrate with the individual securement members bonded thereto to the first assembly station, wherein the ear substrate with the individual securement members comprises third registration features;
detecting positions of the third registration features using a third sensor;
cutting the ear substrate into individual ear pieces based on the positions of each of the third registration features,
wherein the chassis comprises fourth registration features;
detecting the positions of the fourth registration features using a fourth sensor; and
bonding the individual ear pieces to the chassis based on a position of the fourth registration features at the first assembly station to form a multi-piece absorbent article.

16. The method of claim 15, wherein the first assembly station comprises:
a first cutting roll;
a first transfer device positioned adjacent the first cutting roll, wherein the first transfer device is configured to advance the individual ear pieces such that the individual ear pieces are accelerated to the first velocity; and
a first bonding roll posited adjacent the first transfer device, wherein the first bonding roll bonds the individual ear pieces to the chassis.

17. The method of claim 15, wherein the second assembly station comprises:
a second cutting roll;
a second transfer roll positioned adjacent the second cutting roll, wherein the second transfer roll is configured to advance the individual securement members toward the ear substrate; and
a second bonding roll positioned adjacent the second transfer roll, wherein the second bonding roll operatively engages the second transfer roll to bond the individual securement members to the ear substrate.

18. A method for assembling a multi-piece absorbent article, the method comprising:
feeding a chassis to a first assembly station at a first velocity, the chassis comprising a topsheet, a backsheet joined to the topsheet, and an absorbent core positioned between the topsheet and the backsheet;
feeding an ear substrate to a second assembly station at a second velocity;
feeding a securement member substrate to the second assembly station at a third velocity,
wherein the securement member substrate comprises first registration features and second registration features;
teaching a first sensor to recognize the first registration features;
identifying the first registration features using the first sensor;
cutting the securement member substrate into individual securement members based on a position of the first registration features;
teaching the first sensor to recognize the second registration features;
identifying the second registration features using the first sensor;
cutting the securement member substrate into individual securement members based on a position of the second registration features;
switching between the first sensor recognizing the first registration features and the first sensor recognizing the second registration features;
bonding the individual securement members to the ear substrate at the second assembly station, wherein each of the individual securement members is positioned with respect to an individual feature on the ear substrate;
cutting the ear substrate into individual ear pieces; and
bonding the individual ear pieces to the chassis at the first assembly station to form a multi-piece absorbent article.

19. The method of claim 18, comprising recalling the first registration features to the first sensor.

20. The method of claim 18, comprising recalling the second registration features to the first sensor.
